(19) United States
(12) Patent Application Publication
(10) Pub. No.: US 2006/0160077 A1
(43) Pub. Date: Jul. 20, 2006

(54) Method for analyzing the spatial distribution of a chemical substance retained by a biological matter

(75) Inventors: Jukka Lausmaa, Goteborg (SE); Peter Sjovall, Molnlycke (SE); Hakan Nygren, Billdal (SE)

Correspondence Address: MERCHANT & GOULD PC, P.O. BOX 2903, MINNEAPOLIS, MN, 55402-0903, US

(73) Assignee: SP SVERGES PROVNINGS OCH FORSKNINGSINSTITUT AB, BORAS (SE)

(21) Appl. No.: 10/516,712

(22) PCT Filed: Jun. 5, 2003
(86) PCT No.: PCT/SE03/00937

§ 371 Date: Dec. 1, 2004

(30) Foreign Application Priority Data
Jun. 5, 2002 (SE) 0201705-1

(51) Int. Cl. C12Q 1/68; G01N 33/53; G01N 33/00

(52) U.S. Cl. 435/6; 435/7.1; 436/86

(57) ABSTRACT

A method of analyzing the spatial distribution of at least one chemical substance retained by a biological matter comprises the steps of (a) supplying a sample of said biological matter as a specimen surface; (b) producing at least one imprint of said specimen surface on at least one corresponding separate substrate surface, said at least one chemical substance being transferred to the same with retained lateral distribution thereon; (c) subjecting said at least one imprint to imaging mass spectrometry, at least one signal from at least two points being produced, the magnitude of said at least one signal being dependent on the amount of said at least one chemical substance laterally present on said substrate surface; (d) recording said at least one signal from said at least two points; and (e) determining said spatial distribution of said at least one chemical substance from said at least one image of said at least one imprint.

The invention concerns a method for identification and localization of chemical substances (biomolecules) in biological samples. More precisely, the invention describes a method of analyzing the presence of a biomolecule within a biological preparation, for example a blood cell or a tissue sample, with preserved information on the loclization of the molceule in question. The new method can be used for global measurements of subcellular dynamics of gene expression, proteins, metabolites etc, the spatial distribution of at least one chemical substance retained by a biological matter being analyzed.

Cellular reactions against allogenic materials involve the production of signal mediators in connection with contact with different materials and drugs. For example, the blood reaction to foreign materials may engage several major defense systems, e.g. the coagulation cascade, the complement system, fibrinolysis, the kinins, platelet derived growth factors, platelet chemokines, and leukocyte derived factors, like prostaglandins, lipid peroxidation products or ceramides. Attempts to measure blood reactions to materials by choosing one of these factors will always meet with the possibility that other factors may be more important. Methods available today for measuring cell reactions comprise immunocytochemistry and the like, one pre-determined substance at a time being detected. In U.S. Pat. No. 6,051,372 a method is shown which is used for making analogues of a template molecule for recognition and specific binding of biomolecules. A structural imprint of a template molecule is made from a mixture of different amphiphiles that can be induced to undergo a phase transition between a highly mobile liquid-like state and a solid-like crystalline state.

However, in connection with for example proteomics it is desirable to be able to apply general global measurements, whereby a large number of components, for example proteins, can be simultaneously detected in one sample only. Global measurements can explain how proteins, nucleic acids, and small molecules interact with each other to form networks or modules that carry out specific functions. Today, such measurements are integrated, i.e. the measurements are performed in liquid media or cell suspensions, large volumes being required with accompanying complicated separation techniques.

Thus there is a strong demand in the rapidly advancing fields of gene expression acquisition technologies, gene expression data analysis, functional analysis of biological control systems, proteomics, modelling and analysis of kinetic networks, metabolomics, signal transduction, morphogenesis, molecular neurobiology, etc, for a method, whereby it is possible to measure several factors simultaneously, rather than by studying the detailed behaviour of single components. Methods for global measurements on individual cells, including subcellular levels, are not available today.

According to the invention, a method is provided for analyzing the spatial distribution of at least one chemical substance retained by a biological matter. The chemical substance should mainly comprise organic material, which for example can comprise a lipid, an amino acid, a peptide, a protein, a carbohydrate, a nucleotide, a transmitter substance, a drug, or a targeting molecule. The biological matter can for example comprise cells, tissue, virus, body liquid, or biological molecules. Thus, the chemical substance retained by the biological matter can be located within or on the same.

In order to determine the spatial distribution of the chemical substance, a targeting molecule could be arranged to bind to or react with specific targeted moieties of known identity of the biological matter and function as a marker for those molecules which are to be identified. For example, when specific proteins is to be analyzed, antibodies or fragments thereof, which are directed towards specific targeted moieties on the same, can be used as targeting molecules. Similarly, when a specific DNA-sequence of a DNA-molecule is to be analyzed, the targeting molecule is a complementary DNA-sequence to the nucleotide of interest. The targeting molecule can also comprise a chemical label, for example an unusual element or an isotope, in order to improve the detectability in the analytical technique employed, i.e. when larger molecules (e.g. whole proteins) are to be detected. In this connection an unusual element or isotope means an element or an isotope which is not naturally present or present only in low concentrations in the biological matter analyzed.

The method according to the invention is general and can be used directly on complicated specimens, such as dialysis membranes after use. For example, peritoneal cells can be analyzed in connection with peritoneal dialysis by supplying the membrane used as specimen surface. Thus, the method is especially useful for studying cell preparations of blood cells on biomaterials. By studying material/blood reactions, information regarding the influence of man-made synthetic products on the cells can be detected directly.

In the inventive method a small amount of biological matter is transferred from a biological preparation to a suitable substrate, a chemical imprint being produced. The substrate with the imprint thereon can then be subjected to imaging analysis by means of known methods, and the spatial distribution of a chemical substance is determined from the image of the imprint.

In order to identify and localize chemical substances retained by a biological matter, several aspects have to be taken into consideration, such as

(i) the amount of transferred material must be controlled, and

(ii) the original spatial distribution of a chemical substance within or on a biological matter must be preserved and retained until the analysis thereof has been performed.

The first step in the inventive method is to supply a sample of the biological matter as a specimen surface. Such a sample can be supplied as a specimen of a solid or semisolid material. An in situ specimen surface can be used directly when for example the healing (ingrowth) of a titanium implant with a structured surface is to be studied.

The sample of the biological matter can also be supplied as a specimen surface by applying it on a solid surface, the solid surface being provided as a support for the biological matter. In this case the biological matter is in a more liquid state, such as blood and tissue fluid, but can also be a more delicate matter, such as a frozen tissue section.

The solid surface is generally a glass surface, but can be any other suitable solid surface in dependence on the specific application. This is especially relevant when cells are to be analyzed for adhesion, spreading or chemotactic movement.

If necessary, the specimen surface can be prepared by subjecting it to lyophilization, freeze-substitution, or air drying.

The biological matter can also be fractured or cut, for example after freezing, by means of known techniques in order to expose its interior before the imprint is produced.

In order to improve the imprinting effect, the specimen surface can be pretreated immediately before the imprint is produced. One pretreatment comprises the condensation of liquid of a non-polar solvent and/or a polar solvent onto the specimen surface. Preferably, the polar solvent is a water solution.

It is preferred that the pretreatment is accomplished by first bringing the specimen surface to room temperature or cooling the same to a lower temperature and then condensing the solvent vapor thereon by arranging the specimen above a heated container containing the liquid. In order to be effective, the imprint should be produced within 100 s after the pretreatment of the specimen surface.

According to the invention, at least one imprint of the specimen surface is then produced on at least one corresponding separate substrate surface, whereby the at least one chemical substance is transferred to the same with retained lateral distribution thereon.

A thin surface layer of the biological sample is transferred to a substrate surface by means of an imprinting process, the substrate surface being pressed against the specimen surface. In this connection, it is important that the spatial distribution of chemical substances present in the biological sample is retained or reflected in the lateral distribution of the transferred material in the imprint on the substrate surface.

Multiple sequential imprints (“replicas”) can also be produced from the same area of the specimen surface. In this case each of the imprints is produced on a separate substrate surface. These imprints should be produced in such a way that monolayers are “peeled off” and transferred to the corresponding substrate surface. Such a procedure facilitates the subsequent imaging analysis of each chemical substance retained by the biological matter with reference to its spatial distribution.

The main advantage of the imprinting step is that the substrate surface can be optimized in order to improve a subsequent analysis:

(i) the specificity of the imaging analysis can be improved, i.e. the possibility to identify specific organic molecules, which otherwise would be difficult or impossible to identify,

(ii) the sensitivity of the subsequent imaging analysis can be improved, i.e. the intensity of the characteristic signals from the selected chemical substances can be improved, thereby making localization at smaller concentration levels possible, and

(iii) the substrate surface may be designed in order to achieve effective immobilization of the analyzed chemical substances, which otherwise may be vaporized or in any other way lost for detection during the analysis (e.g. due to vacuum conditions during analysis).

A suitable surface, such as a metal surface, should be provided in order to be compatible with the analytical technique employed in the inventive method. Preferred metal substrate surfaces are silver, gold, palladium, platinum, nickel, chromium, and copper.

In addition, the substrate surface should be polished and/or cleaned immediately before the imprint is produced. Suitable cleaning methods are chemical etching, plasma cleaning, or UV/ozone treatment. Of course, the cleaning methods can be combined.

Immediately prior to imprinting a thin layer of metal can be deposited onto the substrate material, for example by means of vapor or electrodeposition. Preferably, the metal is silver.

Thus, a crucial step in the inventive method is the production of the imprint of the specimen surface on the substrate surface in order to transfer and “immobilize” chemical substance(s) with retained distribution.

The imprint is preferably produced by pressing the specimen surface against the substrate surface. This can be accomplished by pressing a compressible material against the opposite side of the specimen surface and/or the opposite side of the substrate surface and by applying thereon a force between 0.01 and 10 MPa. The pressing should be performed for up to 100 s.

In this process individual components, such as ions and larger molecules, are transferred to the substrate surface. An imprint with retained distribution is obtained, which is dependent on the pretreatment and pressing parameters.

The pressing procedure is facilitated if the specimen and/or the substrate is flexible, i.e. made of a flexible material.

Likewise, the transfer of chemical substance(s) to the substrate surface is facilitated by the substrate surface being structured. Preferably, the substrate surface is structured with protrusions of 0.01-5 μm in width and/or length.

Finally, the distribution of the at least one chemical substance is determined by subjecting the imprints on each substrate surface to imaging mass spectrometry (ion microscopy). This is accomplished by producing at least one signal from at least two points of the substrate surface. The magnitude of this signal is dependent on the amount of the chemical substance laterally present on the substrate surface. Mass spectra are obtained with high mass resolution as well as images with high lateral resolution. The resolution is between 100 nm and 1 μm.

Preferably, such signals recorded from these at least two points are recorded from an array of points on the substrate surface. The signals can also be recorded from at least two points which originate from subsequent imprints on separate substrate surfaces.

Each image is in turn produced from these signals, the colour or the brightness in each point of the image being dependent on the magnitude of the signal from the corresponding point on the substrate surface. In this way images of chemical distributions are obtained. The analysis as well as the regeneration of images is accomplished by means of advanced information technology, whereby image processing as well as statistics for handling and processing of the large amounts of data is provided.

Thus, an imaging chemical analysis is performed on the imprinted substrate surface, images being provided which show the lateral distribution of selected chemical substances on the imprinted substrate surface. This, in turn, reflects the spatial distribution of that chemical substance on the biological sample.

A suitable imaging mass spectrometry is a Secondary Ion Mass Spectrometry (SIMS). This is a surface analytical technique that has been employed for spatially resolved analysis of atoms and molecules at the single cell and subcellular levels.

With this technique silver is the preferred substrate surface, since silver is an almost optimal substrate for the analysis of intact molecular ions because of the ability of silver to cationize large molecules.

Thus, when deposited on a clean silver substrate the chemical substance(s) can be cationised by Ag⁺, peaks in the spectrum being provided which correspond to the mass of the intact molecule plus the Ag⁺ ion (M+Ag)⁺. A conclusive identification of the detected molecules is then possible. The identification of unknown compounds is aided by spectral matchings with a library.

For the cationization of the chemical substance by substrate ions to occur in SIMS, the chemical substance to be analyzed must not be present on the substrate surface in too large quanitites. Thus, the pressing is performed so that the imprint represents below 5 monolayers, preferably below 2 monolayers, comprising the chemical substance(s) on the substrate surface. In this connection a monolayer is a monomolecular “film” of native individual molecular components which are transferred without substantial conformational changes in their structure.

Preferably, a focused beam of ions should be produced by the primary ion source in the SIMS, the ions being C₆₀, Ga, In, or Au ions. When gold ions are used, they are clusters of n ions, in which n≦10. The focused beam should have a diameter below 10 μm, preferably below 1 μm.

Preferably, the secondary ion mass spectrometry is Time of Flight-Secondary Ion Mass Spectometry (TOF-SIMS). This is a mass spectrometric method with a high lateral resolution of down to 60 nm combined with the ability to measure secondary ions with masses up to at least 10 000 atomic mass units.

This type of secondary ion mass spectrometry is a relatively new technique for chemical surface analysis and it has several advantages compared to other surface analysis methods. Most significantly, TOF-SIMS is the only method which has the potential for spatially resolved identification and chemical analysis of organic molecules on surfaces in the submicrometer range.

When using TOF-SIMS as the imaging analysis method, the imprint is preferably made on a metal surface, most preferably on a silver surface. When an imprinted silver surface is analysed, unfragmented organic molecules can be detected due to the formation of silver cationized ion complexes, thereby making identification of the imprinted organic substances possible.

In order to improve the formation of cationied ion complexes during the image analysis, the biological matter can be subjected to a salt solution before and/or after the sample of biological matter has been supplied as a specimen surface, the salt being transferred to the substrate surface during the production of the imprint. The salt solution can be a sodium salt, a potassium salt, a copper salt or a silver salt solution. Preferably a silver salt solution is used.

Likewise, in order to improve the formation of cationied ion complexes a thin layer of metal, preferably silver, can be deposited by means of vapor deposition onto the substrate after the imprint has been produced. In this case the layer of metal should have a thickness of less than 100 nm.

A TOF-SIMS spectrum is recorded under high vacuum by scanning the primary ion beam over the area of interest on the substrate surface and acquiring a positive or negative mass spectrum of the ions leaving the surface.

The imaging mass spectrometry can also be a Matrix Assisted Laser Desorption Ionization (MALDI). Preferably, Matrix Assisted Laser Desorption Ionization-Time of Flight Mass Spectrometry (MALDI-TOF) is used. In this case a light sensitive matrix is applied onto the substrate surface before and/or after the production of the imprint.

A light sensitive matrix can also be applied onto the specimen surface before the imprint is produced. When the imprint is produced, a portion of the light sensitive matrix will then be transferred to the substrate surface.

The light sensitive matrix can be α-cyano-4-hydroxycinnamic acid, trans-3-indoleacrylic acid, 3-methoxy-4-hydroxycinnamic acid, 2,5-dihydroxybenzoic acid, or 3,4-dihydroxycinnamic acid.

The light source of the MALDI should comprise a focused laser beam, preferably an ultraviolet laser beam.

### EXAMPLES

The invention will now be further described and illustrated by reference to the following examples. It should be noted, however, that these examples should not be construed as limiting the invention in any way.

### Example 1

### Ion Microscopy

Ions or other molecules from dried specimens are transferred to and immobilized on a silver surface by carefully pressing a freshly etched silver foil onto the dried specimen surface.

For analysis of organic compounds according to the invention, the accumulated primary ion dose is kept below the so called static limit, which means that the analysis is completed before the analyzed surface has been significantly damaged by the primary ions. In a TOF-SIMS image, the brightness of each pixel reflects the signal intensity of a selected ion in that pixel. The recorded TOF-SIMS spectra are stored in raw data files which contain complete spatial and spectral information from the data collection, thereby allowing for subsequent extraction of images of arbitrary ions and extraction of mass spectra from restricted areas within the analysis area at any time after data collection. Data are collected at either high mass resolution m/Δm >7000 or lateral resolution (<100 nm).

### Example 2

### Whole Blood In Vitro

Venous blood from a volunteer is sampled and placed in drops onto different material surfaces and incubated at 37° C. in a humid chamber for varied periods of time. The coagulated blood is then gently washed off and the surfaces are allowed to dry in air. Each preparation is washed with distilled water and dried.

The result of this procedure is a surface layer of plasma proteins and blood cells. The blood cells adhere and are activated differently at different surfaces by detecting the cell expression of integrins and selectins.

More specifically, capillary blood was placed in drops on a clean glass surface and incubated for 30 min at 37° C. The clot was rinsed off with Dulbeccos phosphate-buffered saline and the saline was removed from the glass surface-adhering cells by a rinse in 0.15M NH4COOH at pH 7.2-7.4. The glasses were then placed on a solid copper block pre-cooled with liquid nitrogen in a vacuum chamber that was evacuated down to 10⁻⁴-10⁻⁵ bar.

### Example 3

### Distribution of Cell Components

A clean silver foil is pressed against a glass surface prepared as above and the imprinted silver foil is subsequently analyzed by means of TOF-SIMS at different mass-to-charge ratios of different ions (m/z) with reference to Na⁺, K⁺, Ca⁺, amino acids, and cholesterol, a resolution of less than 0.5 μm being obtained.

1) Distribution of m/z=23 (Indicative of Na⁺).

In this case the resulting TOF-SIMS images showed platelets with a low internal concentration of Na+ and leukocytes (see below) with membrane leakage of Na⁺.

2) Distribution of m/z=30 (Indicative of CH₄N⁺).

This signal is common for several different amino acids, their presence being established.

3) Distribution of m/z=39 (Indicative of K⁺).

Platelets exhibit a high internal concentration of K⁺, indicating an intact membrane, whereas the leukocytes exhibit membrane leakage of K⁺.

4) Distribution of m/z=40.1 (Indicative of Ca⁺).

All cells exhibit a granular distribution of Ca⁺.

5) Distribution of m/z=493.3 and m/z=495.3 (Indicative of Cholesterol-¹⁰⁷Ag⁺ and Cholesterol-¹⁰⁹Ag⁺, Respectively) and m/z=879.6 and m/z=881.6 (Indicative of Cholesterol Dimer-¹⁰⁷Ag⁺ and Cholesterol Dimer-¹⁰⁹Ag³⁰, Respectively).

These combined distributions resulted in a very reliable localization of cholesterol in the cells studied.

Thus, the imaging of subcellular distribution can be demonstrated at a resolution better than 500 nm for signals corresponding to Na⁺, K⁺, Ca⁺⁺, cholesterol and total protein.

### Example 4

### Cell Preparation

Three different cell preparation methods have been used, air drying, freeze substitution, and freeze drying (lyophilization). Air drying was performed in saline followed by rinsing with water or volatile buffers to remove salts. The presence of salt always ruined all possibilities to obtain reproducible data. Freeze substitution was performed in ethanol, acid ethanol, methanol, acid methanol, methanol/water 80/20 in various combinations of buffers and volatile salts. The use of solvents, even dilute methanol, always removed cholesterol from the cytoplasmic membranes.

The only preparation method that gave reproducible localisation of membrane lipids was freeze drying in volatile salts. Cholesterol, cholesterol dimer and phosphocholine have been localised. Cholesterol and phosphocholine showed different and apparently complementary localisation in surface-adhering leukocytes.

### Example 5

### Platelet Adhesion and Activation

The adhesion and spreading of platelets on protein-coated surfaces is studied with respect to receptors involved and membrane expression of integrins and selectin. The spreading of cells is often accompanied by changes in membrane composition e.g. the exposure of phosphatidylserine in the outer leaflet seen during apoptosis. Such an exposure of other membrane lipids, also with a short halftime due to extracellular breakdown, is studied by means of the method according to the invention.

### Example 6

### Chemotaxis

Chemotaxis, defined as the ability of orientation and directed migration in chemical gradients, is a key response of the immune system and a universal cell biological phenomenon. The regulation of this process is complex and not characterized in detail. The compartmentalization of the intracellular signaling system in chemotaxis is a key issue in understanding the mechanisms that control cell orientation in chemotactic gradients. The spatial intracellular resolution of the cell components provide data with reference to those mechanisms, especially to time resolution.

In this connection the inositol lipids (PIP2 and PIP3) are important lipid intracellular messengers under study that are involved in the local control of the actin cytoskeleton and they have distinct functions in the local and global regulation of pseudopode formation. Other lipid mediators, such as diacyl glycerol, are involved in secretory responses, such as degranulation and superoxide release, are also studied.

Glass surfaces are first coated with different proteins by means of physical adsorption, a routinely used technique. Freshly isolated cells are then incubated at the protein-coated surfaces. For experiments with chemotaxis, special chambers have been constructed for exposure of the cells with a gradient of a chemoattractant. Cell adhesion, polarisation and spreading is studied by means of the method according to the invention and compared with fluorescence microscopy.

Accordingly, the inventive method (“ion microscopy”) can be used as a tool In cell biology and enables the analysis and localisation of cell signal mediators, like phospholipids, lipid oxidation products, and ultimately large molecules like whole proteins. The global molecular distribution of individual components within a cell can be reproduced in order to obtain cell specific information on subcellular dynamics of gene expression and proteins.

The method is also applicable to cell surface interactions as well as the influence of different drugs on cell reactions. A comparison can be performed before and after the biological matter has been exposed to different environmental factors. In addition, new materials can be studied, which are developed for the treatment of wounds, dialysis and implants.

1. A method of analyzing the spatial distribution of at least one chemical substance retained by a biological matter, characterized by the steps of
(a) supplying a sample of said biological matter as a specimen surface;
(b) producing at least one imprint of said specimen surface on at least one corresponding separate substrate surface, said at least one chemical substance being transferred to the same with retained lateral distribution thereon;
(c) subjecting said at least one imprint to imaging mass spectrometry, at least one signal from at least two points being produced, the magnitude of said at least one signal being dependent on the amount of said at least one chemical substance laterally present on said substrate surface;
(d) recording said at least one signal from said at least two points; and
(e) determining said spatial distribution of said at least one chemical substance from said at least one image of said at least one imprint.

2. The method as in claim 1, wherein said at least one chemical substance mainly comprises organic material.

3. The method as in claim 2, wherein said organic material comprises a lipid, an amino acid, a peptide, a protein, a carbohydrate, a nucleotide, a transmittor substance, a drug, or a targeting molecule.

4. The method as in claim 3, wherein said nucleotide is a DNA-molecule.

5. The method as in claim 3, wherein said targeting molecule is a complementary DNA-sequence.

6. The method as claim 1, wherein said targeting molecule is an antibody or a fragment thereof.

7. The method as in claim 3, wherein said targeting molecule comprises a chemical label.

8. The method as in claim 7, wherein said chemical label is an unusual element or an isotope.

9. The method as in claim 1, wherein said biological matter comprises cells, tissue, virus, body liquid, or biological molecules.

10. The method as in claim 1, wherein said sample of said biological matter is supplied as a specimen surface in situ.

11. The method as in claim 1, wherein said sample of said biological matter is supplied as a specimen surface by applying it on a solid surface.

12. The method as in claim 11, wherein said solid surface is a glass surface.

13. The method as in claim 1, wherein multiple sequential imprints are produced from the same area of said specimen surface.

14. The method as in claim 1, wherein said biological matter is fractured or cut in order to expose its interior before producing said at least one imprint.

15. The method as in claim 1, wherein said specimen surface is pretreated immediately before producing said at least one imprint.

16. The method as in claim 15, wherein said specimen surface is pretreated by condensing a liquid of a non-polar solvent and/or a polar solvent onto the same.

17. The method as in claim 16, wherein said polar solvent is a water solution.

18. The method as in claim 16, wherein said specimen surface is first brought to room temperature or cooled and is then arranged above a heated container containing said liquid.

19. The method as in claim 1, wherein said at least one imprint is produced within 100 s after said pretreatment of said specimen surface.

20. The method as in claim 1, wherein said specimen and/or said substrate is flexible.

21. The method as in claim 1, wherein said substrate surface is a metal surface.

22. The method as in claim 21, wherein said metal is silver, gold, palladium, platinum, nickel, chromium, or copper, preferably silver.

23. The method as in claim 1, wherein said substrate surface is structured.

24. The method as in claim 23, wherein said substrate surface is structured with protrusions of 0.01-5 μm.

25. The method as in claim 1, wherein said substrate surface is polished.

26. The method as in claim 1, wherein said substrate surface is cleaned immediately before producing said at least one imprint.

27. The method as in claim 26, wherein said substrate surface is cleaned by means of chemical etching, plasma cleaning, or UV/ozone treatment, or a combination thereof.

28. The method as in claim 1, wherein said specimen surface is subjected to lyophilization, freeze-substitution, or air drying before producing said at least one imprint.

29. The method as in claim 1, wherein said biological matter is subjected to a salt solution before and/or after supplying said sample of biological matter as a specimen surface.

30. The method as is claim 29, wherein said salt is a sodium salt, a potassium salt, a copper salt or a silver salt, preferably a silver salt.

31. The method as in claim 1, wherein said at least one imprint is produced by pressing said specimen surface against said substrate surface.

32. The method as in claim 31, wherein said pressing is accomplished by means of a compressible material.

33. The method as in claim 31, wherein said pressing is accomplished by applying a force between 0.01 and 10 MPa.

34. The method as in claim 31, wherein said pressing is performed for up to 100 s.

35. The method as in claim 31, wherein said pressing is performed so that said at least one imprint represents below 5 monolayers, preferably below 2 monolayers, comprising said at least one chemical substance on said substrate surface.

36. The method as in claim 21, wherein a metal layer is deposited onto said substrate surface before producing said at least one imprint.

37. The method as in claim 1, wherein a metal layer is deposited onto said substrate surface after producing said at least one imprint.

38. The method as in claim 37, wherein said layer of metal has a thickness of less than 100 nm.

39. The method as in claim 36, wherein said layer of metal is a silver layer.

40. The method as in claim 1, wherein said imaging mass spectrometry is a Secondary Ion Mass Spectrometry.

41. The method as in claim 40, wherein said Secondary Ion Mass Spectrometry is Time of Flight-Secondary Ion Mass Spectometry.

42. The method as in claim 40, wherein a focused beam of ions is produced by the primary ion source in said Secondary Ion Mass Spectrometry.

43. The method as in claim 42, wherein said ions are C₆₀, Ga, In, or Au ions.

44. The method as in claim 43, wherein said Au ions are clusters of n ions, n≦10.

45. The method as in claim 42, wherein said focused beam has a diameter below 10 μm, preferably below 1 μm.

46. The method as in claim 1, wherein a light sensitive matrix is applied onto said substrate surface before producing said at least one imprint.

47. The method as in claim 1, wherein a light sensitive matrix is applied onto said substrate surface after producing said at least one imprint.

48. The method as in claim 1, wherein a light sensitive matrix is applied onto said specimen surface before producing said at least one imprint, a portion of said light sensitive matrix being transferred to the substrate surface when said at least one imprint is produced.

49. The method as in claim 1, wherein said imaging mass spectrometry is a Matrix Assisted Laser Desorption Ionisation.

50. The method as in claim 49, wherein the light source of said Matrix Assisted Laser Desorption Ionization comprises a focused laser beam, preferably an ultraviolet laser beam.

51. The method as in claim 1, wherein said at least one signal is recorded from an array of points on said substrate surface.

52. The method as in claim 1, wherein said at least one image is produced from said at least one signal, the colour or the brightness in each point of said at least one image being dependent on the magnitude of said at least one signal from the corresponding point on said substrate surface.
